(19) United States
(12) Patent Application Publication
(10) Pub. No.: US 2006/0052387 A1
(43) Pub. Date: Mar. 9, 2006

(54) Organic compounds

(76) Inventor: Clay B Marsh, Upper Arlington, OH (US)

Correspondence Address: CALFEE HALTER & GRISWOLD, LLP, 800 SUPERIOR AVENUE, SUITE 1400, CLEVELAND, OH, 44114, US

(21) Appl. No.: 10/519,654

(22) PCT Filed: Jun. 26, 2003
(86) PCT No.: PCT/US03/20279

§ 371 Date: Aug. 19, 2005

Related U.S. Application Data
(60) Provisional application No. 60/391,633, filed on Jun. 26, 2002.

(51) Int. Cl. A61K 31/496

(52) U.S. Cl. 514/252.18

(57) ABSTRACT

4-(4-methylpiperazin-1-ylmethyl)-N-[4-methyl-3-(4-pyridin-3-yl)pyrimidin-2ylamino)phenyl]benzamide of the formula (I): or a pharmaceutically acceptable salt thereof for the treatment of inflammation.

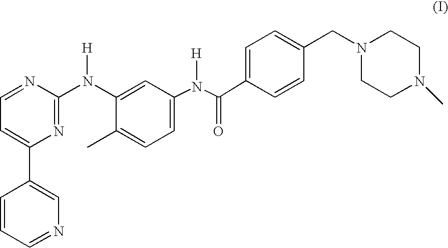

### CROSS-REFERENCE TO RELATED APPLICATIONS

This application claims priority to U.S. Provisional Patent Application Ser. No. 60/391,633, filed on Jun. 26, 2002, the entirety of which is incorporated herein by reference.

### FIELD OF THE INVENTION

The invention relates to the use of 4-(4-methylpiperazin-1-ylmethyl)-N-[4-methyl-3-(4-pyridin-3-yl)pyrimidin-2-ylamino)phenyl]-benzamide (hereinafter “COMPOUND I”) or a pharmaceutically acceptable salt thereof for the manufacture of pharmaceutical compositions for use in the reduction of inflammation, to the use of COMPOUND I or a pharmaceutically acceptable salt thereof in the reduction of inflammation, to a method of treating warm-blooded animals including mammals, especially humans suffering from or susceptible to inflammation by administering to a said animal in need of such treatment an effective dose of COMPOUND I or a pharmaceutically acceptable salt thereof.

The monomethanesulfonic acid addition salt of COMPOUND I (hereinafter “SALT I”) and a preferred crystal form thereof are described in PCT patent application WO 99/03854 published on Jan. 28, 1999, incorporated herein by reference.

### BACKGROUND

Inflammation is the general term for the local accumulation of fluid, plasma proteins and white blood cells that is initiated by physical injury, infection, or a local immune response. Acute inflammation is the term used to describe early and often transient episodes, while chronic inflammation occurs when the infection persists or during autoimmune responses. Many different forms of inflammation are seen in different diseases.

Monocytes are produced in the bone marrow and are circulating cells constituting about 5% of the total white blood cells. They usually circulate in the bloodstream for 24-48 hours. In the absence of growth factors, the circulating monocytes die of apoptosis. The growth factor macrophage colony stimulating factor (M-CSF) is important in monocyte survival. Monocytes are responsible for the production of inflammatory cytokines IL-1 and TNF and are involved in many inflammatory diseases, particularly chronic inflammatory diseases.

### SUMMARY OF THE INVENTION

The present invention provides methods of reducing or preventing inflammation in a subject, the method comprising administering an anti-inflammatory effective amount of 4-(4-methylpiperazin-1-ylmethyl)-N-[4-methyl-3-(4-pyridin-3-yl)pyrimidin-2-ylamino)phenyl]-benzamide of Compound I 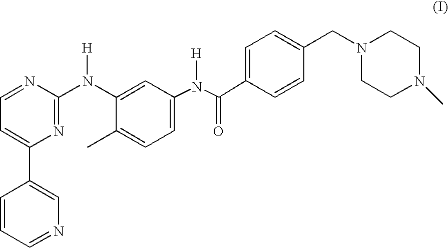
or a pharmaceutically acceptable salt thereof to a subject in need of such treatment. One preferred class of pharmaceutically acceptable salts of Compound I are acid addition salts. A preferred pharmaceutically acceptable salt of Compound I is a monomethanesulfonate salt.

The method of treating subjects having inflammation using Compound I or a pharmaceutically acceptable salt thereof is useful for treating subjects having inflammation caused by any source. In one embodiment, the method comprises administering an anti-inflammatory effective amount of Compound I or a pharmaceutically acceptable salt thereof to subjects having inflammation involving monocytes. In another embodiment, the method comprises administering an anti-inflammatory effective amount of Compound I or a pharmaceutically acceptable salt thereof to subjects having inflammation involving macrophage colony stimulating factor (M-CSF)-stimulated monocytes. In another embodiment, the method comprises administering an anti-inflammatory effective amount of Compound I or a pharmaceutically acceptable salt thereof to subjects having inflammation, wherein such inflammation can cause such conditions as autoimmune diseases, arthritis, transplant-associates rejections, and lung injuries.

The anti-inflammatory effective amount of the 4-(4-methylpiperazin-1-ylmethyl)-N-[4-methyl-3-(4-pyridin-3-yl)pyrimidin-2-ylamino)phenyl]-benzamide or pharmaceutically acceptable salt thereof is in the range from 10 mg to 1000 mg, preferably in the range from 50 mg to 600 mg, and may be administered one or more times daily.

Further provided are uses of Compound I in the treatment of inflammation in subjects, particularly mammalian subjects, and preferably human subjects. The use of compound I may be for the treatment of any inflammation and is preferably for inflammation involved with monocytes and/or involving macrophage stimulating factor (CSF)-stimulated monocytes. Further provided is the use of Compound I or a pharmaceutically acceptable salt thereof for treatment of inflammation related to autoimmune diseases, arthritis, and lung injuries.

Further provided are methods of using 4-(4-methylpiperazin-1-ylmethyl)-N-[4-methyl-3-(4-pyridin-3-yl)pyrimidin-2-ylamino)phenyl]-benzamide or pharmaceutically acceptable salt thereof to suppress the differentiation of monocytes. In accordance with the present invention, the suppression of monocytes may occur either in vivo or in vitro.

### DETAILED DESCRIPTION OF THE INVENTION

It has now surprisingly been demonstrated that COMPOUND I or pharmaceutically acceptable salt thereof can significantly reduce the survival of M-CSF-stimulated monocytes in vitro.

Incubation of monocytes stimulated with M-CSF with increasing amount of SALT I blocks the monocyte survival response to M-CSF stimulation in a SALT I-dose dependent manner. SALT I significantly increases DNA fragmentation and caspase-3 activation in M-CSF stimulated monocytes, it also reduces the number of monocytes surviving at 18 hours.

COMPOUND I is 4-(4-methylpiperazin-1-ylmethyl)-N-[4-methyl-3-(4-pyridin-3-yl)pyrimidin-2-ylamino)phenyl]-benzamide having the formula I

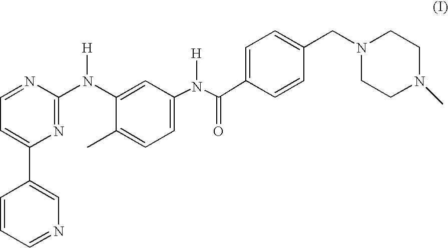

The invention relates to the use of COMPOUND I or a pharmaceutically acceptable salt thereof for the manufacture of a medicament for the treatment of inflammation.

The present invention also relates to the use of COMPOUND I or a pharmaceutically acceptable salt thereof for the manufacture of a medicament for the treatment of inflammation involving monocytes.

The present invention relates also to the use of COMPOUND I or a pharmaceutically acceptable salt thereof for the manufacture of a medicament for the treatment of inflammation involving M-C SF-stimulated monocytes.

In one embodiment of the invention, COMPOUND I or a pharmaceutically acceptable salt thereof is used for the manufacture of medicament for suppressing the differentiation of monocytes, especially in inflammatory diseases.

In a preferred embodiment of the invention the inflammatory diseases comprise autoimmune diseases, arthritis, lung injuries.

By “treating” is meant curing, ameliorating reducing, or tempering the severity of the inflammation or the symptoms associated therewith. The terms “treating,” “treatment,” and “therapy” as used herein refer to curative therapy, prophylactic therapy, and preventative therapy.

The terms “therapeutically effective” and “pharmacologically effective” are intended to qualify the amount of 4-(4-methylpiperazin-1-ylmethyl)-N-[4-methyl-3-(4-pyridin-3-yl)pyrimidin-2-ylamino)phenyl]-benzamide of Compound I or pharmaceutically acceptable salt thereof that, over absence of treatment, will achieve the goal of improvement in healing, particularly reducing inflammation, in a subject suffering from an inflammation. The 4-(4-methylpiperazin-1-ylmethyl)-N-[4-methyl-3-(4-pyridin-3-yl)pyrimidin-2-ylamino)phenyl]benzamide of Compound, I is useful in the treatment of chronic and acute inflammation regardless of the cause of the inflammation. Compound I is especially useful in the treatment of inflammation involving (i.e. associated with or shown to be caused by) monocytes. Compound I is also especially useful in the treatment of inflammation involving macrophage-colony stimulating factor (M-CSF)-stimulated monocytes.

As used herein, “inflammation” and “inflammatory disease” are meant to encompass any inflammation in a subject regardless of cause. The inflammation may be, but is not necessarily, involved with, or causally related with monocytes, white blood cells, macrophages, or macrophage colony stimulating factor (M-CSF). Additionally, “inflammation” and “inflammatory disease” also encompass both acute and chronic inflammatory conditions. Some non-limiting examples of inflammation include coronary artery diseases, autoimmune diseases, arthritis, transplant-associates rejections, lung injuries, atherosclerosis, and pulmonary fibrosis. The 4-(4-methylpiperazin-1-ylmethyl)-N-[4-methyl-3-(4-pyridin-3-yl)pyrimidin-2-ylamino)phenyl]-benzamide of Compound I or pharmaceutically acceptable salt thereof may be used to alleviate inflammation in the subject as a short-term or long-term treatment, or may be prophylactic, as to suppress atherosclerosis or pulmonary fibrosis.

The term “subject” for purposes of treatment includes any human or animal subject who has experienced, is experiencing, or is at risk of developing chronic or acute inflammation. In addition to being useful for human treatment, the compounds of the present invention are also useful for veterinary treatment of mammals, including companion animals and farm animals, such as, but not limited to dogs, cats, horses, cows, sheep, and pigs. Preferably, subject means a human.

Pharmaceutically acceptable salts of COMPOUND I are pharmaceutically acceptable acid addition salts, like for example with inorganic acids, such as hydrochloric acid, sulfuric acid or a phosphoric acid, or with suitable organic carboxylic or sulfonic acids, for example, aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid; or amino acids, such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic acid, or aromatic sulfonic acids, for example, benzene-, p-toluene- or naphthalene-2-sulfonic acid.

Another aspect of the invention provides pharmaceutical compositions, for medical use, comprising 4-(4-methylpiperazin-1-ylmethyl)-N-[4-methyl-3-(4-pyridin-3-yl)pyrimidin-2-ylamino)phenyl]-benzamide of Compound I or pharmaceutically acceptable salt thereof in combination with an acceptable carrier therefor and optionally with other therapeutically-active ingredients or inactive accessory ingredients. The carrier must be pharmaceutically-acceptable in the sense of being compatible with the other ingredients of the formulation and not deleterious to the recipient. The pharmaceutical compositions include those suitable for oral, topical, inhalation, rectal or parenteral (including subcutaneous, intramuscular and intravenous) administration.

Formulations of the present invention suitable for oral administration may be presented as discrete units such as capsules, cachets, tablets, boluses or lozenges, each containing a predetermined amount of the active compound; as a powder or granules; or in liquid form, e.g., as an aqueous solution, suspension, syrup, elixir, emulsion, dispersion, or the like.

Formulations suitable for parenteral administration conveniently comprise a sterile preparation of the active compound in, for example, water for injection, saline, a polyethylene glycol solution and the like, which is preferably isotonic with the blood of the recipient.

Useful formulations also comprise concentrated solutions or solids containing the 4-(4-methylpiperazin-1-ylmethyl)-N-[4-methyl-3-(4-pyridin-3-yl)pyrimidin-2-ylamino)phenyl]-benzamide of Compound I, or pharmaceutically acceptable salt thereof, which upon dilution with an appropriate solvent give a solution suitable for parenteral administration.

Preparations for topical or local applications comprise aerosol sprays, lotions, gels, ointments, suppositories etc., and pharmaceutically-acceptable vehicles therefore such as water, saline, lower aliphatic alcohols, polyglycerols such as glycerol, polyethylene glycerol, esters of fatty acids, oils and fats, silicones, and other conventional topical carriers. In topical formulations, the subject compounds are preferably utilized at a concentration of from about 0.1% to 5.0% by weight.

In addition to the aforementioned ingredients, the formulations of this invention may further include one or more optional accessory ingredient(s) utilized in the art of pharmaceutical formulations, i.e., diluents, buffers, flavoring agents, colorants, binders, surface active agents, thickeners, lubricants, suspending agents, preservatives (including antioxidants) and the like.

Preferably, the mode of administration of the 4-(4-methylpiperazin-1-ylmethyl)-N-[4-methyl-3-(4-pyridin-3-yl)pyrimidin-2-ylamino)phenyl]-benzamide of Compound I, or pharmaceutically acceptable salt thereof, will be oral.

Depending on species, age, individual condition, mode of administration and the clinical picture in question, effective doses of SALT I, for example, corresponding to daily doses of the active substance (free base) of about 10-1000 mg, preferably 50-600 mg, especially 100400 mg, are administered to warm-blooded animals of about 70 kg bodyweight. For adult patients with inflammatory diseases a starting dose of, e.g., 200 mg daily can be recommended. For patients with an inadequate response after an assessment of response to therapy with 200 mg daily, dose escalation can be safely considered and patients may be treated as long as they benefit from treatment and in the absence of limiting toxicities.

The invention relates also to a method for administering to a human subject suffering from an inflammatory disease, COMPOUND I or a pharmaceutically acceptable salt thereof, which comprises administering a pharmaceutically effective amount of COMPOUND I or a pharmaceutically acceptable salt thereof to the human subject, e.g., once daily for a period exceeding 3 months. The invention relates especially to such method wherein a daily dose of SALT I corresponding to 10-1000 mg, especially 50-600 mg, preferably 100-400 mg of the active substance (free base) is administered.

The unit dosage form contains 10-200 mg, most preferably 50-150 mg of the monomethanesulfonate salt of 4-(4-methylpiperazin-1-ylmethyl)-N-[4-methyl-3-(4-pyridin-3-yl)pyrimidin-2-ylamino)phenyl]-benzamide of the formula I.

Importantly, the clinical toxicity profile of oral SALT I therapy was shown to be remarkably favorable, SALT I is relatively low toxic.

The experimental procedures and the materials of the following examples are found in Kelley et al., J. Biol. Chem., Vol. 37, pp. 26393-26398 (1999), incorporated herein by reference.

### EXAMPLE 1

### SALT I Reduces the Survival of M-CSF-Stimulated Human Monocytes

Monocytes (3×10⁶ cells/condition) were incubated in 5% FBS/RPMI 1640 with dimethylsulfoxyde (DMSO, a control vehicle) or with 20 ng/mL of M-CSF and DMSO or with 20 ng/mL of M-CSF and increasing concentrations of SALT I (1-100 NM) for 24 hours. Monocytes were incubated with SALT I or DMSO for one hour prior to the addition of M-CSF. The cells were counted in 5 blinded, high-powered fields. The data represents 3 independent experiments and are given as numbers of viable cells after the incubation period.

| DMSO | + | + | + | + | + | + | + |
|---|---|---|---|---|---|---|---|
| M-CSF | − | + | + | + | + | + | + |
| SALT I in pM | − | − | 1 | 5 | 10 | 50 | 100 |
| Experiment 1 | 154 | 228 | 130 | 156 | 138 | 46 | 15 |
| Experiment 2 | 54 | 36 | 79 | 47 | 46 | 7 | 11 |
| Experiment 3 | 48 | 121 | 143 | 75 | 58 | 36 | 16 |
| Mean | 85 | 148 | 117 | 93 | 81 | 30 | 14 |
| S.D. | 46 | 93 | 25 | 42 | 38 | 15 | 2 |
| S.E.M | 26 | 54 | 15 | 24 | 22 | 9 | 1 |

Monocyte survival was suppressed in M-CSF treated cells incubated with SALT I (1-100 NM) versus cells incubated with M-CSF in the presence of DMSO.

### EXAMPLE 2

### SALT I Induces DNA Fragmentation Events in M-CSF Stimulated Monocytes

Cells can die by undergoing programmed cell death also called apoptosis. The hallmark of this type of cell death is the fragmentation of nuclear DNA into small fragments of about 200 base pairs.

Human monocytes (3×10⁶/mL, 1 mL/condition) were incubated for 18 hours in 5% fetal bovine serum (FBS)/RPMI 1640 with M-CSF (20 ng/mL) added 1 hour after DMSO (solvent control) or SALT I (10 or 100 NM) was added. The cells were also incubated without stimulus. Apoptotic DNA fragments were purified from the monocytes samples using a DNA isolation kit (Suicide-Track DNA isolation kit, Oncogene Research Products, Cambridge, Mass.) Small molecular weight cytosolic DNA isolated fragments and a 123 by DNA ladder for size comparison were run on the same 4% agarose gel. Two independent experiments give the comparable results (data not shown). SALT I at doses of 10 and 100 μM induced DNA fragmentation and its export to the cytosol in contrast with monocytes stimulated with M-CSF or DMSO.

### EXAMPLE 3

### SALT I Induces Caspase-3 Activity in M-CSF-Stimulated Human Monocytes

Caspase-3 is a cysteine aspartate protease involved in the cascade leading to DNA fragmentation. Caspase-3 cleaves one subunit of a dimer called DNA fragmentation factor (DFF) at a tetrapeptide sequence DEVD, the other subunit then activates a nuclease that degrades DNA.

Monocytes (3×10⁶/mL, 2 mL/condition) were lysed fresh (FRESH) or incubated for 18 hours with DMSO or with 20 ng/mL of M-CSF and DMSO or with 20 ng/ml of M-CSF and SALT I (0.1-100 NM). M-CSF was added to the monocytes media one hour after DMSO or SALT I. Cells were lysed in KPM buffer by freeze and thaw method, the protein was equalized and caspase-3 activity determined. Caspase-3 activity is defined as the amount of free amino trifluoromethylcoumarin (AFC) released from the caspase-3 selective fluorosubstrate DEVD-AFC measured by fluorometry. In the following table, caspase activity is given in pmol/min/mg.

| DMSO | − | + | + | + | + | + | + |
|---|---|---|---|---|---|---|---|
| M-CSF | − | − | + | + | + | + | + |
| SALT I in pM | − | − | − | 0.1 | 1 | 5 | 10 |
| Donor 1 | 14 | 550 | 92 | 156 | 356 | 573 | 629 |
| Donor 2 | 9 | 487 | 131 | 220 | 299 | 329 | 407 |

SALT I significantly increased caspase-3 activation in a SALT I dose-dependent manner.

### EXAMPLE 4

### Freshly Isolated Monocytes Appear to Express Abelson Tyrosine Kinase (Ab1)

Monocytes were tested for the presence of the Ab1. AN is inhibited by SALT I and its presence in monocytes might provide an elucidation of the mechanism undergoing the efficiency of SALT I in inhibiting the M-CSF induced survival of monocytes.

Monocytes (3×10⁶/mL, 10 mUcondition) were isolated and lyzed. The monocytes lysates were immunoprecipitated with polyclonal antibodies or isogenic immunoglobulin G (IgG). Proteins were separated using polyacrylamide gel electrophoresis, transferred to a membrane and immunoblotted using a monoclonal anti-Ab1 antibody. The results (data not shown) showed that Ab1 is present in normal human monocytes.

### EXAMPLE 5

### SALT I Decreases AktR Osphorylation

Akt1 also called protein kinase B is a serine/threonine kinase involved in a signaling pathway activated by the Bcr-Ab1 activity. Phosphorylated Akt1 phosphorylates cellular proteins and promotes cell proliferation and cell survival.

In vitro Akt kinase assays were performed as described in Kelley et al., supra. Briefly, monocytes were incubated overnight with M-CSF (20 ng/mL), serum-starved for 1.5 hours. SALT I (1 or 5 NM) or DMSO (vehicle control) was added, one hour later DMSO and SALT I samples were stimulated with M-CSF (100 ng/mL) for three minutes prior to lysis with Akt lysis buffer. Akt protein was immunoprecipitated from the cell lysate. The blot was probed with anti-phospho-threonine-Akt, stripped and re-probed for total Akt.

### EXAMPLE 6

### Capsules with 4-[(4-methyl-1-piperazin-1-ylmethyl)-N-[4-methyl-3-[[4-(3pyridinyl)-2pyrimidinyllaminolphenyllbenzamide methanesulfonate, β-crystal form

Capsules containing 119.5 mg of the compound named in the title (=SALT I) corresponding to 100 mg of COMPOUND I (free base) as active substance are prepared in the following composition:

Composition

| | | |
|---|---|---|
| SALT I | 119.5 | mg |
| Cellulose MK GR | 92 | mg |
| Crospovidone XL | 15 | mg |
| Aerosil 200 | 2 | mg |
| Magnesium stearate | 1.5 | mg |
| | 230 | mg |

The capsules are prepared by mixing the components and filling the mixture into hard gelatin capsules, size 1.

### EXAMPLE 7

### Capsules with 4-[(4-methyl-1-piperazin-1-ylmethyl)-N-[4-methyl-3-[[4-(3-pyridinyl)-2-pyrimidinyl]amino]phenyl]benzamide methanesulfonate β-crystal form

Capsules containing 119.5 mg of the compound named in the title (=SALT I) corresponding to 100 mg of COMPOUND I (free base) as active substance are prepared in the following composition:

Composition

| | | |
|---|---|---|
| Active substance | 119.5 | mg |
| Avicel | 200 | mg |
| PVPPXL | 15 | mg |
| Aerosil | 2 | mg |
| Magnesium stearate | 1.5 | mg |
| | 328 | mg |

The capsules are prepared by mixing the components and filling the mixture into hard gelatin capsules, size 1.

1. A method for reducing inflammation in a subject, the method comprising administering an anti-inflammatory effective amount of 4-(4-methylpiperazin-1-ylmethyl)-N-[4-methyl-3-(4-pyridin-3-yl)pyrimidin-2-ylamino)phenyl]-benzamide of Compound I 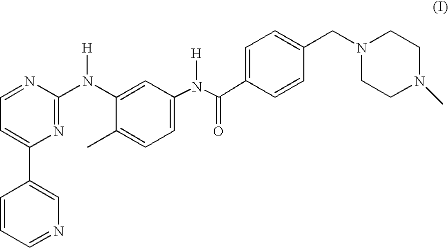
or a pharmaceutically acceptable salt thereof to the subject in need of such treatment.

2. The method of claim 1 wherein the pharmaceutically acceptable salt of 4-(4-methylpiperazin-1-ylmethyl)-N-[4-methyl-3-(4-pyridin-3-yl)pyrimidin-2-ylamino)phenyl]-benzamide of formula I is an acid addition salt.

3. The method of claim 1 wherein the pharmaceutically acceptable salt of 4-(4-methylpiperazin-1-ylmethyl)-N-[4-methyl-3-(4-pyridin-3-yl)pyrimidin-2-ylamino)phenyl]-benzamide of formula I is a monomethanesulfonate salt.

4. The method of claim 1 wherein the inflammation involves monocytes.

5. The method of claim 4 wherein the inflammation involves macrophage colony stimulating factor (M-CSF)-stimulated monocytes.

6. The method of claim 4 wherein the inflammation is involved in conditions selected from the group consisting of autoimmune diseases, arthritis, and lung injuries.

7. The method of claim 1 wherein the inflammation is chronic.

8. The method of claim 1 wherein the inflammation is acute.

9. The method of claim 1 wherein the subject is a mammal.

10. The method of claim 9 wherein the subject is a human subject.

11. The method of claim 1 wherein the anti-inflammatory effective amount of 4-(4-methylpiperazin-1-ylmethyl)-N-[4-methyl-3-(4-pyridin-3-yl)pyrimidin-2-ylamino)phenyl]-benzamide or pharmaceutically acceptable salt thereof is in the range from 10 mg to 1000 mg.

12. The method of claim 11 wherein the anti-imflammatory effective amount is in the range from 50 mg to 600 mg.

13-27. (canceled)

28. The method of claim 2 wherein the inflammation involves monocytes.

29. The method of claim 28 wherein the inflammation involves macrophage colony stimulating factor (M-CSF)-stimulated monocytes.

30. The method of claim 28 wherein the inflammation is involved in conditions selected from the group consisting of autoimmune diseases, arthritis, and lung injuries.

31. The method of claim 3, wherein the inflammation involves monocytes.

32. The method of claim 31 wherein the inflammation involves macrophage colony stimulating factor (M-CSF)-stimulated monocytes.

33. The method of claim 31 wherein the inflammation is involved in conditions selected from the group consisting of autoimmune diseases, arthritis, and lung injuries.

34. The method of claim 2 wherein the anti-inflammatory effective amount of 4-(4-methylpiperazin-1-ylmethyl)-N-[4-methyl-3-(4-pyridin-3-yl)pyrimidin-2-ylamino)phenyl]-benzamide or pharmaceutically acceptable salt thereof is in the range from 10 mg to 1000 mg.

35. The method of claim 3 wherein the anti-inflammatory effective amount of 4-(4-methylpiperazin-1-ylmethyl)-N-[4-methyl-3-(4-pyridin-3-yl)pyrimidin-2-ylamino)phenyl]-benzamide or pharmaceutically acceptable salt thereof is in the range from 10 mg to 1000 mg.
